(19) United States
(12) Patent Application Publication
(10) Pub. No.: US 2025/0134838 A1
(43) Pub. Date: May 1, 2025

(54) DERMAL WAXING PATCH

(71) Applicant: Maggy François, Bowie, MD (US)

(72) Inventor: Maggy François, Bowie, MD (US)

(21) Appl. No.: 18/495,393

(22) Filed: Oct. 26, 2023

(51) Int. Cl. A61K 31/167; A61K 9/70; A61K 31/045; A61K 31/60; A61K 36/61

(52) U.S. Cl. CPC A61K 31/167; A61K 9/7038; A61K 31/045; A61K 31/60; A61K 36/61

(57) ABSTRACT

A transdermal anesthetic skin patch is laid onto to the skin for topical administration of an anesthetic chemical composition. Topical route administration of the chemical composition is the delivery system of the compound to the skin surface. The skin patch may be triangular in shape.

### FIELD

The present disclosure generally relates to a dermal waxing skin patch, and particularly related to a releasable anesthetic skin patch.

### BACKGROUND

The waxing industry is a thriving industry and an important part of the economy. Currently, the U.S. waxing salon industry is currently worth more than $6.9 billion, a testament to the success of the waxing salon industry. Waxing has become an integral part of the beauty industry and an extremely lucrative business venture.

Waxing is performed both on men and women. The waxes are performed both at home and at a salon by a professional. There are many types of waxes ranging from a bikini line to an extensive Brazilian wax which is full hair removal of the lower pelvic region. More recently, individuals are waxing different areas of the body including the legs, arms, back, underarms, face, and stomach. The type of wax an individual receives depends on their personal preferences and how much hair they want to be removed.

Waxing is a hair removal method that involves applying hot wax to the skin and then quickly removing it, along with the hair, in one swift motion. Despite its popularity as a hair removal method, waxing can be an incredibly painful process for many individuals. The sensation of hot wax being applied to the skin can be uncomfortable and even painful, particularly in more sensitive areas. When the wax is quickly removed, it can cause a sharp, stinging sensation that can be difficult to tolerate, particularly for those with lower pain thresholds. The pain associated with waxing can be further exacerbated by factors, such as the thickness and coarseness of the hair, the individual's skin sensitivity, and the skill of the waxing technician. While some individuals may find waxing to be a tolerable experience, for many others, the pain associated with waxing can be a significant deterrent to using this hair removal method.

### SUMMARY

The following summary presents a simplified summary of certain features. The summary is not an extensive overview and is not intended to identify key or critical elements.

An aspect of the present disclosure includes a transdermal-anesthetic patch system for delivering a therapeutic anesthetic composition to a dermis of a human. An aspect of the present disclosure relates to a triangular skin patch for topically anesthetizing the human dermis. One configuration manifests an article of the present disclosure as a flat triangular shaped white sheet which resembles the layer of a dermal patch upon which a chemical numbing composition may be adhered in or to thereof.

An aspect of the present disclosure relates to a skin patch for topically anesthetizing the human dermis, the patch includes three layers-a skin fabric; a chemical anesthetic composition topically anesthetizing the human dermis for preventing the transmission of pain signals to the brain and producing the desired local anesthesia effect; and a plastic film abutting the composition. In accordance with the teachings of this present disclosure, transdermal anesthetic application, upon the removal of the anesthetized patch the individual may experience reduced perceived pain levels for enabling a waxing operation to be more of comfortable experience to the human subject.

An aspect of the present disclosure, includes a topical application of dermal waxing patch on the human figure. An aspect of the present disclosure provides a skin fabric with anesthetic adhesive that is laid on the skin.

An aspect of the present disclosure includes an article of the manufacture in the form of a square patch to indicate the use of the patch in various forms for different areas of the body in addition to the most popular genital areas. In yet another aspect of the present disclosure the anesthetic agents form one or more chemical compounds of an anesthetic component of a transdermal patch.

An aspect of the present disclosure includes a transdermal-anesthetic patch system may have a backing layer, an anesthetic-compound layer; and a release liner attached to an anesthetic-compound layer in which the anesthetic-compound layer includes a skin releasable adhesive matrix; about 1.0% w/w Menthol; about 0.50% w/w Menthol Salicylate; and about 4.0% w/w Lidocaine.

An aspect of the present disclosure includes a transdermal-anesthetic patch system may have a backing layer, an anesthetic-compound layer; and a release liner attached to an anesthetic-compound layer, in which the anesthetic-compound layer includes a skin releasable adhesive matrix; about 0.50% w/w Menthol Salicylate; and about 4.0% w/w Lidocaine.

An aspect of the present disclosure includes a transdermal-anesthetic patch system for delivering a therapeutic anesthetic composition to a dermis of a human. The patch may have a backing layer, an anesthetic-compound layer; and a release liner attached to the anesthetic-compound layer\, in which the anesthetic-compound layer includes a skin releasable adhesive matrix; about 1.0% w/w Menthol; and about 4.0% w/w Lidocaine. In yet another aspect, a transdermal-anesthetic patch includes an anesthetic-compound layer further having about 10.0% w/w Benzocaine. In yet another aspect, a transdermal-anesthetic patch system includes an anesthetic-compound layer further having Aloe Vera. In yet another aspect, a transdermal-anesthetic patch includes an anesthetic-compound layer further having Melaleuca oil. In yet another aspect, a transdermal-anesthetic patch includes an anesthetic-compound layer further having Vitamin E.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. As used in the specification and in the claims, the singular form of ‘a’, ‘an’, and ‘the’ include plural referents unless the context clearly dictates otherwise.

### BRIEF DESCRIPTION OF THE DRAWINGS

The foregoing and other features and advantages will be apparent from the following more particular description of exemplary constructions of the disclosure. The drawings are not necessarily to scale, with emphasis instead being placed upon the principles of the disclosure.

FIG. 1 shows a view of a transdermal anesthetic skin patch on a human body “A” according to teachings of the present disclosure.

FIG. 2 shows a view of a transdermal anesthetic skin patch according to teachings of the present disclosure.

FIG. 3 shows a view of the release layer of the transdermal anesthetic skin patch according to teachings of the present disclosure.

FIG. 4 shows a schematic cross-sectional view of the layers of the transdermal anesthetic skin patch taken along line 4-4 of FIG. 3 according to teachings of the present disclosure;

FIG. 5 shows a view of the skin anatomy layers according to teachings of the present disclosure.

FIG. 6 shows a view of a square-shaped transdermal anesthetic skin patch to be applied to different areas on a human body.

FIG. 7 shows a view of tables of chemical ingredients composing a transdermal anesthetic skin patch according to teachings of the present disclosure.

FIG. 8 shows a view of transdermal anesthetic skin patches on a human body “A” according to teachings of the present disclosure.

FIG. 9 shows a view of one example table of chemical ingredients composing a transdermal adhesive matrix according to teachings of the present disclosure.

### DETAILED DESCRIPTION

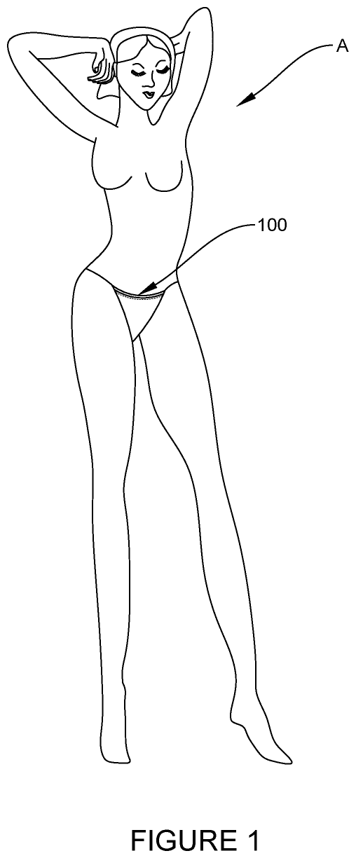

As illustrated through FIGS. 1-9, in accordance with various constructions, a triangular skin patch 100 provides for numbing the lower pelvic region of the human body, in addition to other bodily areas in preparation for a waxing procedure. Within the following description of the illustrative embodiments, there is reference to the accompanying drawings, in which is shown through illustration. It should be understood that other embodiments can be utilized and modified while remaining within the scope of the present disclosure.

As used herein, a CAS Registry Number (also referred to CAS Number) is a unique identification number assigned by the Chemical Abstracts Service (CAS) USA to chemical substance described in the open scientific literature. CAS Registry Numbers can be convenient for database searches. They offer a reliable, common and international link to every specific substance across the various nomenclatures and disciplines used by branches of science, industry, and regulatory bodies. Almost all molecule databases today allow searching by CAS Registry Number.

In the present disclosure, there is provided an anesthetic-compound layer 120 that comprises one or more local anesthetic agents and is capable of delivering a therapeutically effective amount of one or more chemical compounds through the skin barrier of a user. In one construction, a skin releasable adhesive matrix provided on the transdermal patches 100, may be infused with Menthol (CAS No. 1490-04-6), Menthol Salicylate (CAS No. 63-36-5), and Lidocaine (CAS No. 137-58-6) that typically produces a soothing or pain-relieving reaction. In accordance with the teachings of the present disclosure, the composition with the matrix may include Menthol Salicylate (CAS No. 63-36-5) at 0.5% w/w, Lidocaine (CAS No. 137-58-6) at 4.0% w/w, and Menthol (CAS No. 1490-04-6) at 1.0% w/w.

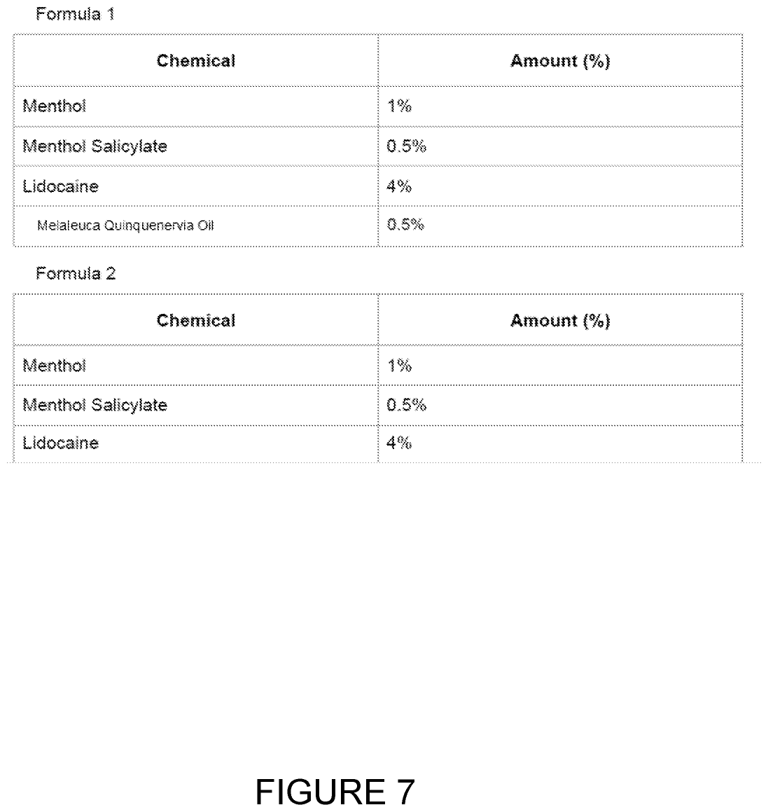

The present disclosure may include Melaleuca oil extract (CAS Nos. 8022-72-8; 85085-48-9; 8022-72-8; 68647-73-4) and Aloe Vera shown (CAS No. 8001-97-6) in FIG. 7 to soothe the skin simultaneous with an anesthetic effect. The wound healing and preventive effects of Aloe Vera is useful to enhance the healing process of dermal injuries after the removal of hair from the waxing procedure. Further, Aloe Vera has anti-inflammatory, skin protection, anti-bacterial, anti-viral, antiseptic properties that can enhance the healing process of the skin after a waxing procedure, particularly in the lower pelvic groin region of a women.

An alternative composition for the patch 100 may include about 4.00% w/w Lidocaine, 1.00% w/w Menthol, 10.00% w/w Benzocaine (CAS No. 94-09-7), Aloe Vera extract (CAS No. 8001-97-6) and less than 1% Vitamin E (CAS No. 1406-18-4). In yet another alternative composition for the patch 100 may include about 4.00% w/w Lidocaine, 1.00% w/w Menthol, Aloe Vera extract (CAS No. 8001-97-6) and less than 1% Vitamin E (CAS No. 1406-18-4).

Another alternative composition for the patch 100 may include about 4.00% w/w Lidocaine, 1.00% w/w Menthol, Aloe Vera extract (CAS No. 8001-97-6). Yet another alternative composition for the patch 100 may include about 4.00% w/w Lidocaine, 1.00% w/w Menthol, and less than 1% Vitamin E (CAS No. 1406-18-4).

An alternative composition for the patch 100 may include about 4.00% w/w Lidocaine, 1.00% w/w Menthol, 10.00% w/w Benzocaine (CAS No. 94-09-7). An alternative composition for the patch 100 may include about 4.00% w/w Lidocaine, 0.5% w/w Menthol Salicylate, 1.00% w/w Menthol, 10.00% w/w Benzocaine (CAS No. 94-09-7), Aloe Vera extract (CAS No. 8001-97-6) and less than 1% Vitamin E (CAS No. 1406-18-4). An alternative composition for the patch 100 may include about 4.00% w/w Lidocaine, 0.5% w/w Menthol Salicylate, 10.00% w/w Benzocaine (CAS No. 94-09-7), and Aloe Vera extract (CAS No. 8001-97-6). An alternative composition for the patch 100 may include about 4.00% w/w Lidocaine, 0.5% w/w Menthol Salicylate, 1.00% w/w Menthol, 10.00% w/w Benzocaine (CAS No. 94-09-7), and less than 1% Vitamin E (CAS No. 1406-18-4).

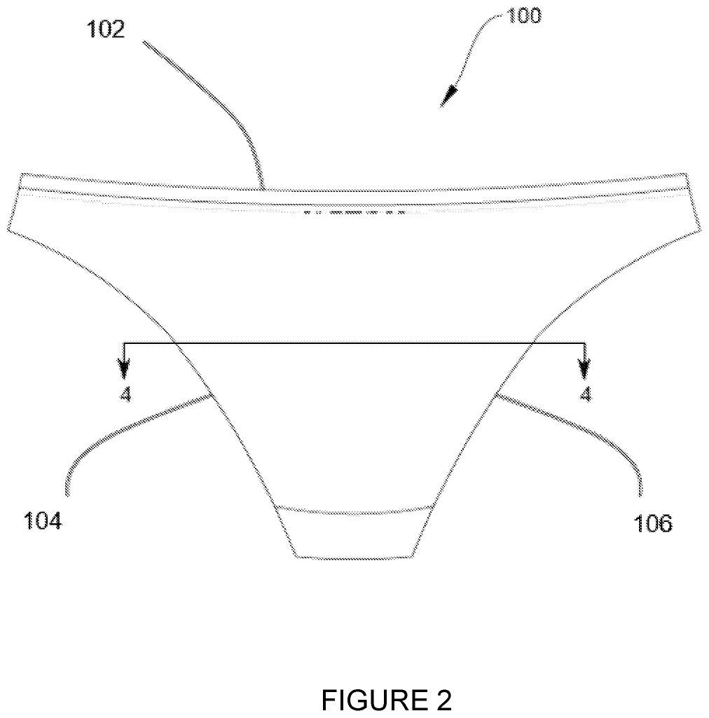

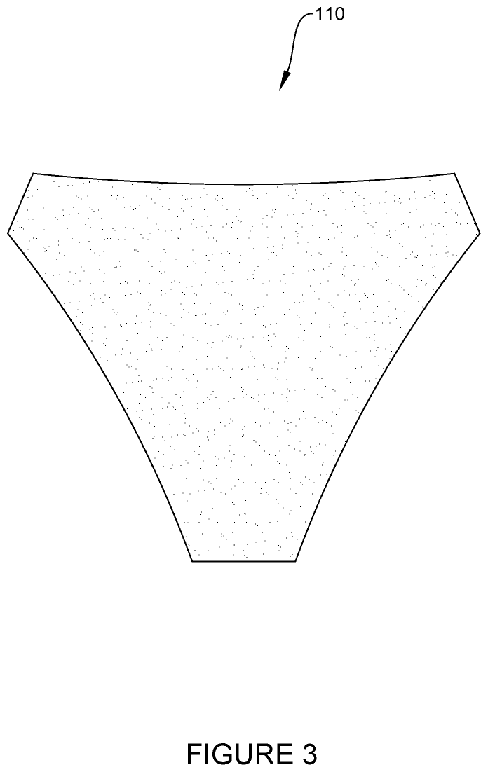

FIG. 1 shows a stylized human body “A” pictured with the skin patch 100 applied to the pelvic private area of the body. Prior to receiving a wax, an individual can mitigate the amount of pain endured during a traditional waxing session by utilizing the transdermal skin patch 100. The triangular shaped patch shown in FIG. 3 can be applied onto the anatomy private areas in the pelvic region to numb and reduce the pain of waxing experiences. The triangular shape shown in FIG. 2 of the patch 100 provides one or more advantages over traditional square, rectangular patches, or topical creams.

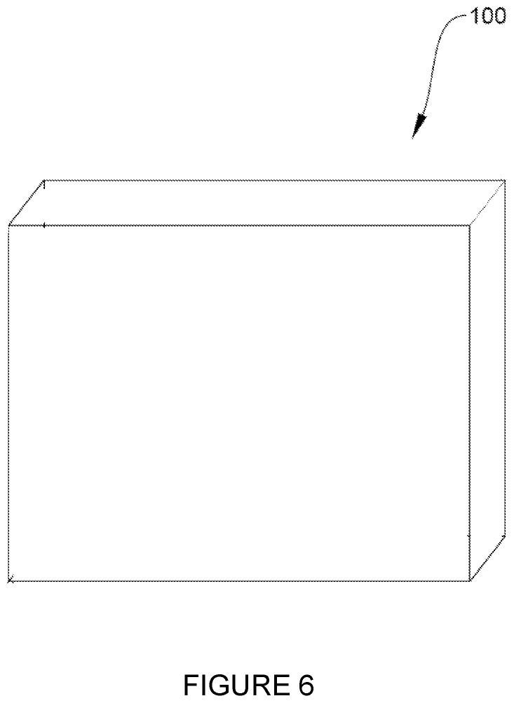

The triangular shape of patch 100 allows for a simpler lay on application in the pelvic private area of the body, reducing the likelihood of wrinkles or folds in the patch which may cause discomfort or uneven anesthetic application. The triangular shape further conforms well to the contours of the lower pelvic region in the groin area, providing a more uniform distribution of the anesthetic chemical compound shown in FIG. 7 over the skin surface. The top part 102, left side 104, and right side 106 of the patch 100 helps assist a smooth surface application to the skin in the pelvic area. The triangular shaped skin patch of the present disclosure also offers versatility for use in other areas of the body as shown in FIG. 1. Referring to FIG. 6, the patch 100 may be cut into square or other shapes to suit the needs of the user.

It should be noted that the dermal patch 100 can be helpful in treating Intertrigo aliment in the lower pelvic region in the groin area. Intertrigo is a reddish rash that appears in skin folds, where skin surfaces rub against each other. The rash can be itchy or painful. Path 100 with anti-inflammatory, skin protection, anti-bacterial, anti-viral, antiseptic properties can enhance the healing process of the skin from the Intertrigo rash. Further Melaleuca oil has anti-bacterial effects helpful in healing the skin from the rash.

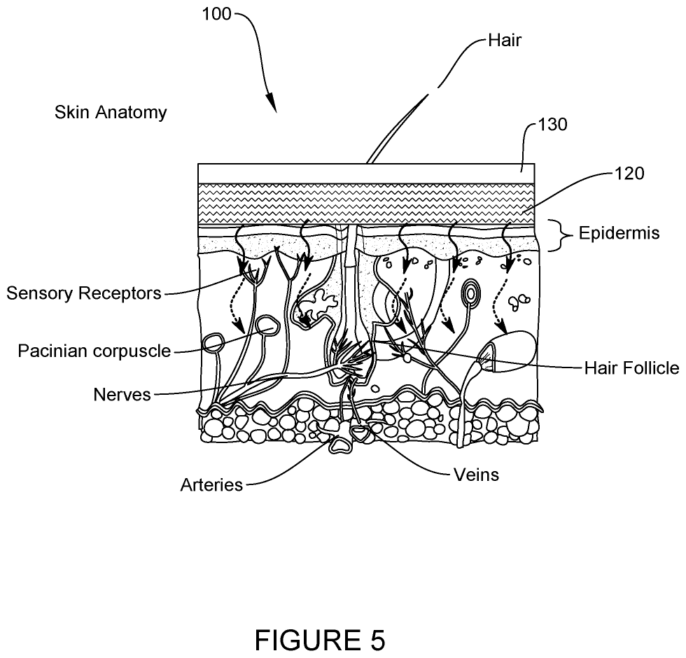

Referring to FIG. 5, the adhesive matrix including the chemical compound(s), targets the area to be waxed infusing into the skin and through the epidermal to provide a soothing, pain-relieving effect to the wearer. In one or more constructions, the adhesive matrix may be selected from a group comprising silicone, polyacrylate copolymers (acrylic) or polyisobutylene (PIB). Chemical compositions of the present disclosure are applied to the skin to elicit one or more of the following effects; (1) an effect on skin surface; (2) an effect within the stratum corneum; or (3) a systematic effect that results from the delivery of sufficient amounts of a chemical composition through the epidermis and the dermis to the micro-vasculature to produce anesthetic systemic concentrations.

The chemical compound matrix of patch 100 has a transdermal delivery that provides the anesthetic effect to the human skin and the underlying tissues and nerves shown in FIG. 5. These compounds are configured to be lipophilic, meaning they have an affinity for fat and other lipids, which are abundant in the skin's outermost layer, the stratum corneum. Referring to FIG. 5, the Stratum Corneum is the outermost layer of the epidermis. This layer is composed of flattened, dead skin cells that are tightly packed together, forming a barrier to prevent the penetration of foreign substances. The chemical compounds of patch 100 are able to penetrate the stratum corneum and enter the deeper layers of the skin. There are several mechanisms by which this can occur, including passive diffusion which is the mechanism by which the present system of patch 100 operates.

With passive diffusion, the chemical compound moves from an area of higher concentration (e.g., the skin patch 100 shown in FIG. 2) to an area of lower concentration; such as the skin shown in FIG. 5. The present disclosure utilizes lipophilic compounds that diffuse more readily through the stratum corneum than hydrophilic compounds because they can dissolve in the lipid-rich environment of the skin thus providing fact acting pain relief to the human subject. Referring to FIG. 7, the composition compounds Lidocaine, Menthol, Menthol Salicylate, Aloe Vera, and Benzocaine referenced herein the present disclosure are lipophilic compounds, for example. The combination of one of more these lipophilic compounds in the concentrations thereof, in addition to the skin soothing agents, such as tea tree provides an outstandingly unique numbing effect to the area to which the patch 100 is applied to in the human body shown in FIG. 1.

Once the anesthetic compound(s) has penetrated the skin, it can bind to receptors in the nerves, preventing the transmission of pain signals to the brain and producing the desired local anesthesia effect. Transdermal delivery of anesthetic compounds can provide an effective and convenient method for producing an anesthetic effect. The chemical compounds as presented herein provides an improved mix of compounds that can reduce the perceived pain levels experienced in waxing as much as 80% to 90% according to some wearers.

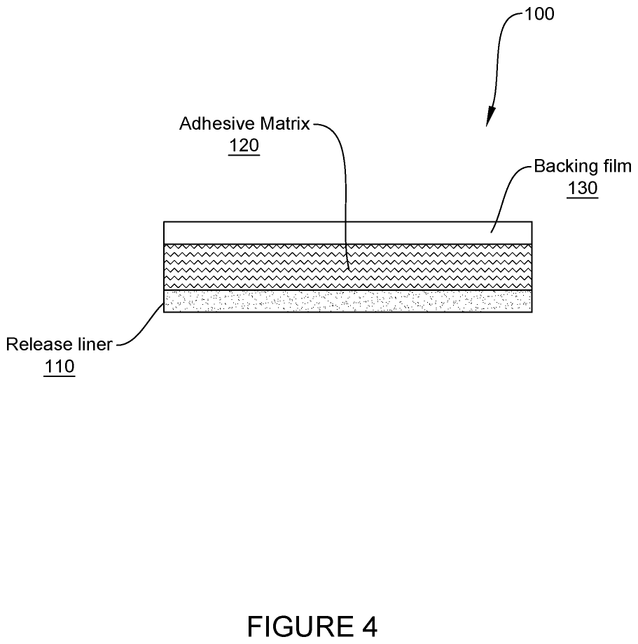

Referring to FIG. 4, the skin patch 100 is configured with three distinct layers that work together to provide an effective and convenient vehicle of delivering the chemical compound to the dermis of a user. The first layer of the skin patch 100 is the protective backing layer in the form of the skin fabric 130, which is selected to protect the adhesive matrix layer (e.g., the anesthetic-compound layer 120) during storage and handling. The anesthetic-compound layer 120 is sandwiched in between the skin fabric 130 and a release liner 110. The anesthetic-compound layer 120 includes one or more anesthetic compounds in a skin releasable adhesive that is configured to adhere to the skin upon application (e.g., pressure sensitive). For example, suitable adhesives can be formulated with skin-friendly adhesives (bio-adhesives), including silicone, polyacrylate copolymers (acrylic), and polyisobutylene (PIB).

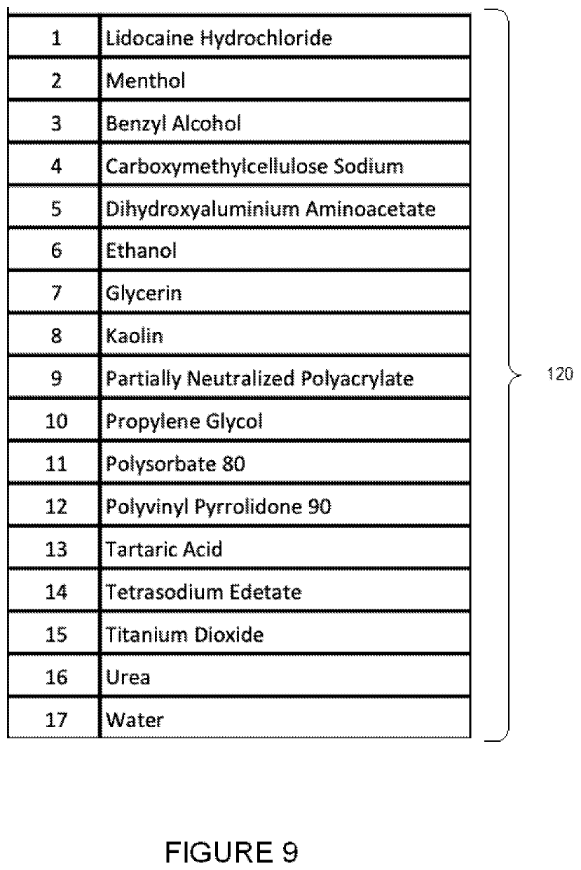

As discussed in the foregoing, the anesthetic-compound layer 120 has a drug-in-adhesive construction in which the dry powder of the active components and excipients may be mixed in with the adhesive during manufacture. One example pressure sensitive adhesive for transdermal system 100 is a commercially available 3M™Acrylic PSA 9811. Another example of a pressure sensitive adhesive for transdermal system 100 is commercially available Dow Corning LIVEO BIO-PSA Silicone Adhesive lines. The adhesives are permeable to many drugs and excipients and are configured to adhere drug-loaded systems to the skin. FIG. 9 shows an example of adhesive matrix compounds used in the present disclosure.

The release liner 110 of the patch 100, may be constructed of a plastic film which acts as a protector to the anesthetic-compound layer, keeping it moist and useful until application. In one use, the plastic film 110 is removed prior to application on the user's skin, exposing the anesthetic-compound layer 120, and allowing for easy placement of the patch onto the desired area of skin.

The three-layer design of the skin patch 100 provides for an effective and convenient means of delivering a topical anesthetic agent, while also providing protection and ease of use. The design also enables for convenient and ease of release of the anesthetic-compound onto the skin surface shown in FIG. 5 ensuring effective localized pain relief.

Still referring to FIG. 4, the skin fabric layer 130 (or backing layer) is configured to provide a surface to which the anesthetic-compound layer can be adhered, providing effective delivery and release of the chemical compound to the skin. The skin fabric 130 is composed of a high-quality, durable fabric material that is capable of withstanding long-term storage of the skin patch. The fabric may be of a woven construction that includes polyester fabric. The fabric material is selected for compatibility with the chemical compound, ensuring that the patch remains intact and effective. One example for the skin fabric is a commercially available 3M™ COTRAN™ Non-Woven Polyurethane Tape.

The release liner 110 is configured to contain the moisture and use of the anesthetic-compound layer 120 therefore the patch can be held long term without the anesthetic-chemical compound layer drying out. This ensures that the patch remains stable and effective until it is applied to the skin. The skin fabric ensures that the patch remains effective over an extended period, allowing for convenient and reliable delivery of the chemical compound ensuring pain relief for the users waxing endeavors.

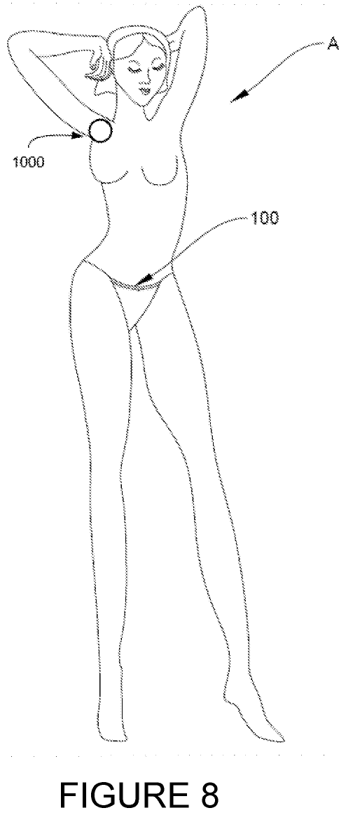

As illustrated in FIG. 8, in accordance with various constructions, a skin patch 1000 provides for numbing under arm of the human body which has sensitive skin, in addition to other bodily areas in preparation for a waxing procedure. The path 1000 includes the feature of patch 100, except for the shape as triangle. Nevertheless, patch 1000 could be used in the pelvic region for a numbing application.

### Example

To evaluate the perceived pain reliving effect of the patch 100, women patients were tested with the patch on areas of skin prior to a waxing procedure. In operation, the film 110 was pulled off the skin fabric 130 to reveal the chemical composition matrix 120 which was applied upon the desired skin area of the body. In subject cases, the patch was topically applied to the skin for a duration of 40-45 minutes, prior to a waxing treatment test skin area. According to some wearers the reduce perceived pain levels experienced in waxing as much as about 80% to 90% compared a skin area with no pain numbing patch.

Terms of degree such as “generally”, “substantially”, “about” and “approximately” as used herein mean a reasonable amount of deviation of the modified term such that the end result is not significantly changed. For example, these terms can be construed as including a deviation of at least +−0.5% of the modified term if this deviation would not negate the meaning of the word it modifies.

Although the compositions and methods of the present specification as well as various commercial and consumer products containing/comprising the same have been described with respect to specific embodiments and examples, it should be appreciated that the present teachings are not limited thereto and other embodiments utilizing the concepts expressed herein are intended and contemplated without departing from the scope of the present teaching as intended in the true spirit and scope of the present disclosure. It is therefore intended any and all modifications, variations, or equivalents that fall within the spirit and scope of the underlying principles are within the scope of this present disclosure and are covered by the appended claims.

What is claimed is:

1. A transdermal-anesthetic patch system for delivering a therapeutic anesthetic composition to a dermis of a human, comprising:
a backing layer,
an anesthetic-compound layer; and
a release liner attached to the anesthetic-compound layer; wherein the anesthetic-compound layer comprises:
a skin releasable adhesive matrix, including:
about 1.0% w/w Menthol;
about 0.50% w/w Menthol Salicylate; and
about 4.0% w/w Lidocaine.

2. The transdermal-anesthetic patch system, according to claim 1, wherein the backing layer further comprises a polyester skin fabric.

3. The transdermal-anesthetic patch system, according to claim 1, wherein the anesthetic-compound layer further comprises about 10.0% w/w Benzocaine.

4. The transdermal-anesthetic patch system, according to claim 1, wherein the anesthetic-compound layer further comprises Aloe Vera.

5. The transdermal-anesthetic patch system, according to claim 1, wherein the anesthetic-compound layer further comprises Melaleuca oil.

6. The transdermal-anesthetic patch system, according to claim 1, wherein the anesthetic-compound layer further comprises Vitamin E.

7. The transdermal-anesthetic patch system, according to claim 2, wherein the anesthetic-compound layer further comprises Aloe Vera.

8. The transdermal-anesthetic patch system, according to claim 3, wherein the anesthetic-compound layer further comprises Aloe Vera.

9. The transdermal-anesthetic patch system, according to claim 3, wherein the anesthetic-compound layer further comprises Melaleuca oil.

10. The transdermal-anesthetic patch system, according to claim 3, wherein the anesthetic-compound layer further comprises Vitamin E.

11. The transdermal-anesthetic patch system, according to claim 4, wherein the anesthetic-compound layer further comprises tea tree.

12. The transdermal-anesthetic patch system, according to claim 11, wherein the anesthetic-compound layer further comprises Vitamin E.

13. The transdermal-anesthetic patch system, according to claim 1, further comprising at least one of a triangular shape and circular shape.

14. The transdermal-anesthetic patch system, according to claim 3, further comprising at least one of a triangular shape and circular shape.

15. The transdermal-anesthetic patch system, according to claim 12, further comprising at least one of a triangular shape and circular shape.

16. A transdermal-anesthetic patch system for delivering a therapeutic anesthetic composition to a dermis of a human, comprising:
a backing layer,
an anesthetic-compound layer; and
a release liner attached to the anesthetic-compound layer; wherein the anesthetic-compound layer comprises:
a skin releasable adhesive matrix, including:
about 1.0% w/w Menthol; and
about 4.0% w/w Lidocaine.

17. The transdermal-anesthetic patch system, according to claim 16, wherein the anesthetic-compound layer further comprises about 10.0% w/w Benzocaine.

18. The transdermal-anesthetic patch system, according to claim 16, wherein the anesthetic-compound layer further comprises about 0.50% w/w Menthol Salicylate and Aloe Vera.

19. The transdermal-anesthetic patch system, according to claim 16, wherein the anesthetic-compound layer further comprises Vitamin E.

20. The transdermal-anesthetic patch system, according to claim 16, further comprising at least one of a triangular shape and circular shape.
